(19) United States
(12) Patent Application Publication
(10) Pub. No.: US 2007/0219583 A1
(43) Pub. Date: Sep. 20, 2007

(54) System And Method For Facilitating Hemostatis With An Absorbable Sponge

(76) Inventors: Eduardo Chi Sing, Dana Point, CA (US); Mark Ashby, Laguna Niguel, CA (US); Tin Tran, Anaheim, CA (US); Richard Greff, St. Pete Beach, FL (US)

Correspondence Address: MILLER, MATTHIAS & HULL, ONE NORTH FRANKLIN STREET, SUITE 2350, CHICAGO, IL, 60606, US

(21) Appl. No.: 10/557,333

(22) PCT Filed: Jun. 14, 2004
(86) PCT No.: PCT/US04/18707

§ 371 Date: Nov. 20, 2006

Related U.S. Application Data
(60) Provisional application No. 60/478,307, filed on Jun. 12, 2003.

(51) Int. Cl. A61B 17/03

(52) U.S. Cl. 606/213

(57) ABSTRACT

The present invention provides for a method and apparatus to provide hemostasis at a blood vessel puncture site, having a hemostasis material and a clot formation accelerator, wherein said clot formation accelerator is substantially dispersed throughout said hemostasis material.

### CROSS-REFERENCE TO RELATED APPLICATIONS

This application claim prioirty to Co-pending U.S. patent appliation Ser. No. 60/478,307, filed Jun. 12, 2003, by inventors Eduardo Chi Sing, Mark Ashby, and Tin Tran, entitiled “Improved System and Method For Facilitating Hemostasis with Absorbable Sponge” which is a continuation-in-part of, and claims priority under 35 U.S.C. §120 to, and incorporates by reference herein in their entirety: 1. Co-pending U.S. patent application Ser. No. 10/421,680, filed Apr. 22, 2002 by inventors Thomas David, Mark Ashby, and Eduardo Chi Sing, entitled “PUNCTURE CLOSURE SYSTEM WITH PIN AND PULL TECHNIQUE”, which is a continuation-in-part of patent application Ser. No. 10/107,539 filed on Mar. 25, 2002, by inventors Andrew H. Cragg, Rodney Brenneman, and Richard Greff, entitled “Apparatus And Method For Percutaneous Sealing Of Blood Vessel Punctures”, which is a divisional of U.S. Pat. No. 6,371,974, filed Aug. 2, 1999, which is a divisional of U.S. Pat. No. 6,071,300 filed Jul. 7, 1997, which is a continuation-in-part of U.S. Pat. No. 5,645,566, filed Sep. 15, 1995; and 2. Co-pending U.S. Patent application Ser. No. 09/966,611, filed Sep. 27, 2001 by inventors Mark Ashby and Eduardo Chi Sing entitled “Absorbable Sponge with Contrasting Agent”, which is a continuation of application Ser. No. 09/630,814, filed Aug. 2, 2000, now abandoned, which is a divisional of U.S. Pat. No. 6,183,497, filed Jun. 17, 1999, which is a continuation-in-part of U.S. Pat. No. 6,071,301, entitled “Device And Method For Facilitating Hemostasis Of A Biopsy Tract”, filed May 1, 1998 by inventors Andrew H. Cragg, Rodney Brenneman, and Mark Ashby.

### FIELD OF THE INVENTION

The present invention relates to an apparatus and method to facilitate hemostasis at a puncture site. More particularly, the present invention relates to a method and apparatus to facilitate hemostasis at a puncture site with clot formation accelerators incorporated within a hemostasis material.

### BACKGROUND OF THE INVENTION

A large number of diagnostic and interventional procedures involve the percutaneous introduction of instrumentation into a vein or artery. For example, coronary angioplasty, angiography, atherectomy, stenting of arteries, and many other procedures often involve accessing the vasculature through a catheter placed in the femoral artery or other blood vessel. Once the procedure is completed and the catheter or other instrumentation is removed, bleeding from the punctured artery must be controlled.

Traditionally, external pressure is applied to the skin entry site to stem bleeding from a puncture wound in a blood vessel. Pressure is continued until hemostasis has occurred at the puncture site. In some instances, pressure must be applied for up to an hour or more during which time the patient is uncomfortably immobilized. In addition, a risk of hematoma exists since bleeding from the vessel may continue beneath the skin until sufficient clotting effects hemostasis. Further, external pressure to close the vascular puncture site works best when the vessel is close to the skin surface but may be unsuitable for patients with substantial amounts of subcutaneous adipose tissue since the skin surface may be a considerable distance from the vascular puncture site.

There are several approaches to close the vascular puncture site including the use of non-absorbable tissue adhesives, absorbable material such as collagen, or anchor and plug systems. To provide for effective clot formation at the vascular puncture site, clot formation accelerators are added to the hemostasis material such as the non-absorbable tissue adhesives, absorbable material, or plug. Clot formation accelerators are currently added by merely surrounding the hemostasis material with the clot formation accelerators. One disadvantage is that the clot formation accelerators, as illustrated in FIG. 2A, may be released too early such as when the hemostasis material enters the tissue tract 210 and is exposed to blood thus forming a clot 212a within the tissue tract. Another disadvantage is that the clot formation accelerators may be easily removed by external factors such as handling of the hemostasis material. Additionally, with the clot formation accelerators located around the hemostasis material, the clot formation accelerator may enter the blood vessel lumen and cause a clot formation 212b within the blood vessel 208. Thus, the blood clot formed is not site specific. Furthermore, clot formations 212c and 212d are formed around the hemostasis material 204, rather than within the hemostasis material 204, which is important since the hemostasis material is located at the vasculature puncture site. Moreover, the clots formed are not effective to provide for hemostasis at the vascular puncture site since it is not well structured nor is it dense enough to facilitate hemostasis.

Thus, there is still a need for a method and apparatus to rapidly facilitate hemostasis at a puncture site with clot formation accelerators incorporated within the matrix of the hemostasis material.

### BRIEF DESCRIPTION OF THE INVENTION

The present invention provides for a method and apparatus to provide hemostasis at a blood vessel puncture site, having a hemostasis material and a clot formation accelerator, wherein said clot formation accelerator is substantially dispersed throughout said hemostasis material.

### BRIEF DESCRIPTION OF THE DRAWINGS

The accompanying drawings, which are incorporated into and constitute a part of this specification, illustrate one or more embodiments of the present invention and, together with the detailed description, serve to explain the principles and implementations of the invention.

In the drawings:

FIG. 1 is a block diagram of a method for forming a clot formation accelerator loaded hemostasis material in accordance with an embodiment of the present invention.

FIG. 2A illustrates the hemostasis material at a puncture site having clot formation accelerator surrounding the exterior of the hemostasis material.

FIG. 2B illustrates the hemostasis material at a puncture site having clot formation accelerator substantially dispersed throughout the hemostasis material.

### DETAILED DESCRIPTION

Embodiments of the present invention are described herein in the context of an improved system and method for facilitating hemostasis with an absorbable sponge. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings. The same reference indicators will be used throughout the drawings and the following detailed description to refer to the same or like parts.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Providing hemostasis at the blood vessel puncture site is important for procedures such as puncture closure. Just as important is the ability to provide for rapid hemostasis at the puncture site and not around the puncture site. Thus, the present invention describes a method and apparatus to efficiently and easily facilitate hemostasis at a puncture site with clot formation accelerators incorporated within and as an intricate part of the matrix of the hemostasis material or gelatin material.

The hemostasis material or gelatin material may be delivered at a blood vessel puncture site through methods which will not be discussed in this application but are further described in detail in: 1. Co-pending U.S. patent application Ser. No. 09/621,670, filed Jul. 24, 2000 by inventors Mark Ashby, Andrew Cragg, Luis Urquidi, Eduardo Chi Sing, and Eric Lee entitled “Depth and puncture control for system for hemostasis of blood vessel”; 2. Co-pending U.S. patent application Ser. No. 10/007,204, filed Nov. 8, 2001 by inventor Mark Ashby entitled “System and method for delivering hemostasis promoting material to a blood vessel puncture site by fluid pressure”; and 3. U.S. Pat. No. 6,086,607 issued to Cragg, et al. Entitled “Device and method for facilitating hemostasis of a biopsy tract”, which are all hereby incorporated by reference herein. The bench tests conducted were conducted using the clot formation accelerator loaded hemostasis material of the present invention in conjunction with different percutaneous delivery systems described in the patent applications and patents above.

The present invention is described with reference to cross-linked gelatin based solutions. The gelatin solution can be used to generate solid pieces of dried gelatin, a foam matrix, or a combination of both. The basic gelatin formulation can be made from between 2%-35% by weight of gelatin, 0.01% to 4.0% by weight of formaldehyde, and water. The manufacturing process of the gelatin is similar to the process described in U.S. Pat. No. 2,465,357 issued to Correll, of which the manufacturing process is incorporated by reference herein in its entirety.

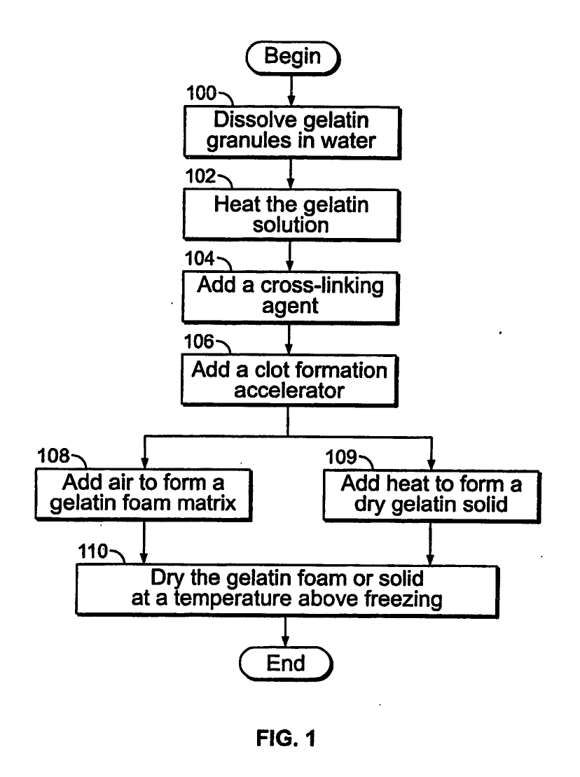

FIG. 1 illustrates a method for forming a clot formation accelerator loaded hemostasis material. At 100, gelatin granules are dissolved in water and heated at 102 and allowed to cool. A cross-linking agent, such as formaldehyde, is added at 104 and mixed with the dissolved gelatin. At 106, a clot formation accelerator is added to the gelatin mixture. Examples of clot formation accelerators are further described below. The mixture may then be formed as a foam matrix or gelatin solid. At 108, air is added to the mixture to create a foam matrix or heat is added to form a dry gelatin solid at 109. The gelatin foam or solid is then dried at a temperature above freezing at 110. Thus, the clot formation accelerator is substantially dispersed within the foam matrix and/or gelatin solid.

Clot formation accelerators may be added by mixing the clot formation accelerators to the gelatin formulation mixture above in different concentrations to form the hemostasis material. Clot formation accelerators may include, but are not limited to, clot agglomeration (via positive charge attraction) such as CaCL₂ or Ca(OH)₂, Chitosan, or thrombogenic agents (bovine Thrombin).

Calcium loaded gelatin components are positively charged and therefore attract negatively charged blood cell resulting in blood clot agglomeration. Materials such as CaCl₂ or Ca(OH)₂ may be used in concentrations ranging from about 0.1M to 1M.

Thrombin is a commonly used thrombogenic agent that affects the clotting cascade directly by causing rapid processing of fibrinogen. The bovine derived thrombin may be obtained in lypophilized powder or solution. The concentration range of thrombin that may be used may be from about 15 units to 50000 units per 250 ml of gelatin solution.

Currently, the use of Thrombin mixed with gelatin inhibits the development and/or stability of the gelatin foam cell structure during the manufacturing process. Two procedures may be used to overcome the degredation of the gelatin foam cell structure. First, the newly produced gelatin foam containing Thrombin may be freeze-dried to prevent gelatin foam cell structure degradation. However, this procedure is costly, inhibits the cross-linking process, and decreases the strength of the gelatin cell structure. Alternatively, sugars and/or polysaccharides may be added to the gelatin and then dried in temperatures above freezing. The use of polysaccharides with the drying process protects and/or blocks the active enzymatic groups on the Thrombin via hydrogen bonding with the active enzymatic groups. This protects the gelatin cell structure from degrading. Sugars and polysaccharides such a sucrose, dextran, and the like may be used.

Chitosan (also known as poly d glucosamine) may also be used since it is a polysaccharide. Tests mixing Chitosan with the Thrombin prior to foaming the solution revealed no degredation of the gelatin foam cell structure and the gelatin was easy to foam after drying in temperatures above freezing. The Chitosan diffused from the foam upon rehydration of the foam which left the Thrombin exposed and active in the gelatin foam. An example of the method is described in detail below.

Chitosan may also be used as a clot formation accelerator. Chitosan, is derived from shellfish and relies on polycation action to achieve rapid clot formation. The concentration amount may range from 0.1 gr to 1 gr per ml of gelatin solution.

The incorporation of a clot accelerator into the hemostasis material as an intricate component of the foam matrix or solid crystalline network, may result in a more effective clot formation relative to the formed clot density and structural integrity, and site specific or boundary limited generation of the puncture site. These clot characteristics may be important when trying to achieve rapid sealing and/or hemostasis at a bleeding or blood vessel puncture site, especially when surrounding tissue configuration, puncture site size, blood pressure and anti-platelet/anticoagulation therapy are factors that a user must consider.

Table 1 and 2 illustrate the configuration of gelatin foam sample generated and tested under similar conditions. Foam samples without clot accelerators were defined as the control and heparinized porcine blood (ACT levels of 300+ seconds) was used to test the hemostatic characteristics of the test samples. The gelatin solution formulation used was: 5% by weight of Gelatin, 0.02% by weight of Formaldehyde, and about 95% water.

TABLE 1
Porcine model

| Clot Accelerator | Concentration | Hemostasis Time |
|---|---|---|
| Control | N/A | 1 minute dwell. 2 minute hold |
| Thrombin | 222 units/ml | 1 minute dwell. No hold |
| Thrombin | 100 units/ml | 1 minute dwell. No hold |

Table 1 results indicate that the hemostasis material or gelatin foam samples with different thrombin concentrations achieve hemostasis after a 1 minute dwell (i.e. when the delivery system is still in place within the tissue tract) without the need of post-procedural manual hold. In contrast, the control sample required a 2 minute post-procedural manual hold, i.e. 2 minutes of manual external compression over the blood vessel puncture site to stop the blood from oozing out of the blood vessel puncture site and prevent the formation of a hematoma.

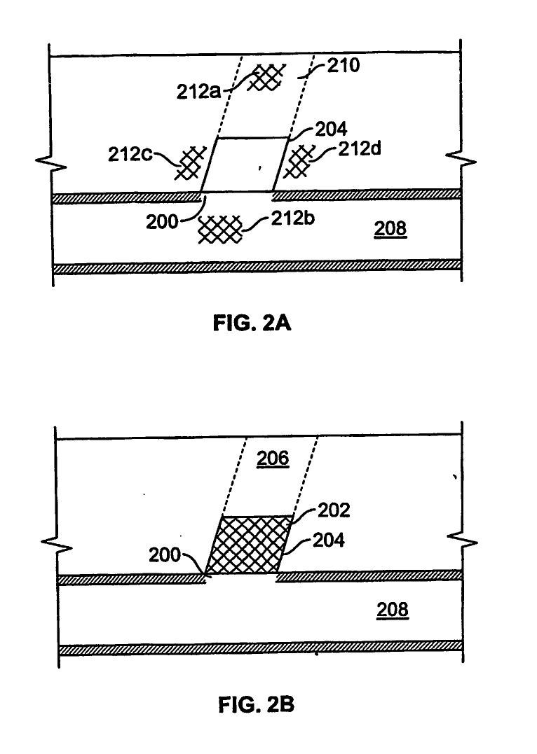

After blood flow was controlled at the puncture site 200, a cut down of the tissue tract revealed a well structured blood clot 202 embedded within the hemostasis material or gelatin foam 204 as illustrated in FIG. 2B. Minimum signs of bleeding along the tissue tract 206 were found which indicate that hemostasis occurred rapidly. No clot formation was found within the blood vessel lumen 208 that indicates that the clot formation accelerator remained within the gelatin foam 204. The gelatin foam 204 was delivered on top of the puncture site 200 in one piece. A cross sectional cut of the delivered gelatin foam revealed blood clot formation throughout the foam. This characteristic is attributed to the blood absorption capability of the foam and its ability to retain the absorbed blood cells within the foam.

TABLE 2
Bench Top Model

| Clot Accelerator | Concentration | Hemostasis Time | |
|---|---|---|---|
| Control | N/A | 120 | sec avg. |
| Thrombin | 222 units/ml | 45 | sec avg. |
| Thrombin | 100 units/ml | 60 | sec avg. |
| Thrombin | 222 units/ml hydration | 75 | sec |
| | solution - surrounding only | | |
| | the outer perimeter of the | | |
| | gelatin foam | | |
| CaCl2 | 0.1M | 60 | sec avg. |
| Ca(OH)2 | 1M | 60 | sec avg. |

Table 2 illustrates the use of clot accelerators Thrombin and Calcium in various concentrations as compared with a control. The results indicate that the foam sample with the highest concentration of thrombin yielded the most concise, dense, and well-structured blood clot in the shortest amount of time. Samples having calcium within its matrix showed similar clot agglomeration characteristics as the foam sample with embedded thrombin in the foam matrix.

The foam sample hydrated with a thrombin hydration solution, only on the outer perimeter of the foam matrix, showed a less dense and less structured blood clot. The clot formation was limited to the outer surface of the foam matrix and signs of small clot formation were observed within the blood and tissue surrounding the gelatin foam.

The above test results indicate that when clot formation accelerators are an intricate component of the gelatin solution or foam matrix, the time to achieve hemostasis at a puncture site is reduced and no post-procedural manual hold or compression is required to effectively seal the bleeding site. Moreover, the clot is formed at the puncture site and not around the puncture site or within the blood vessel. Additionally, the physical characteristics of the formed clot are better suited to achieve effective hemostasis at the puncture site since the clot has a higher density and structural integrity.

The present invention may be used with various other applications such as at the proximal end of a dissolvable or detachable tips as further described in co-pending patent application Ser. No. 10/461,587, filed Jun. 12, 2003 by inventors Mark Ashby, Eduardo Chi Sing, and Tin Tran, entitled “DISSOLVABLE CLOSURE DEVICE” which is incorporated herein by reference in its entirety.

For exemplary purposes only and not intended to be limiting, an Example of the method for forming a clot formation accelerator loaded hemostasis material will be provided.

### EXAMPLE

0.42 g of Chitosan, 17.5 g of gelatin, and 112.5 ml of water were mixed together (“Chitosan mixture”). A solution of 0.7 cc of formaldehyde and 1.7 ml of sodium hydroxide was mixed together and incubated for two hours (“solution”). The Chitosan mixture was added to the solution and heated. 15000 units of Thrombin was then added to the mixture and mixed for about 15 minutes. Measurements conducted at this time revealed that the temperature was 34° C. and the pH was 5.56. The mixture was then mixed for two minutes to obtain the gelatin foam and heated in an oven at 90° F. for forty-eight hours. The gelatin was easy to foam and the gelatin foam cell structure did not degrade.

While embodiments and applications of this invention have been shown and described, it would be apparent to those skilled in the art having the benefit of this disclosure that many more modifications than mentioned above are possible without departing from the inventive concepts herein. The invention, therefore, is not to be restricted except in the spirit of the appended claims.

What is claimed is:

1. An apparatus to provide hemostasis at a blood vessel puncture site, comprising:
a hemostasis material; and
a clot formation accelerator, wherein said clot formation accelerator is substantially dispersed throughout said hemostasis material.

2. The apparatus of claim 1 wherein said clot formation accelerator is a clot agglomeration.

3. The apparatus of claim 1 wherein said clot formation accelerator is Chitosan.

4. The apparatus of claim 1 wherein said clot formation accelerator is a thrombogenic agent.

5. The apparatus of claim 4 further comprising a polysaccharide.

6. The apparatus of claim 6 wherein said polysaccharide is Chitosan.

7. An apparatus to provide hemostasis at a blood vessel puncture site, comprising:
a hemostasis material;
a clot formation accelerator; and
a polysaccharide,
wherein said clot formation accelerator and said polysaccaride are substantially dispersed throughout said hemostasis material.

8. The apparatus of claim 7 further comprising a cross-linking agent.

9. The apparatus of claim 7 wherein said clot formation accelerator is a thrombogenic agent.

10. The apparatus of claim 7 wherein said polysaccharide is Chitosan.

11. An apparatus to provide hemostasis at a blood vessel puncture site, comprising:
a hemostasis material;
a cross-linking agent;
a polysaccharide; and
a clot formation accelerator,
wherein said cross-linking agent, said clot formation accelerator, and said polysaccharide are substantially dispersed throughout said hemostasis material.

12. The apparatus of claim 11 wherein said clot formation accelerator is a thrombogenic agent.

13. The apparatus of claim 11 wherein said polysaccharide is Chitosan.

14. The apparatus of claim 11 wherein said cross-linking agent is a formaldehyde.

15. A method for forming a clot formation accelerator loaded hemostasis material, comprising:
heating gelatin granules in water;
adding a cross-linking agent;
mixing a clot formation accelerator to the cross-linking agent and heated gelatin solution; and
adding air to form a gelatin foam hemostasis material matrix,
wherein said clot formation accelerator is substantially dispersed throughout said hemostasis material.

16. The method of claim 15 wherein said dissolving further comprises adding a polysaccharide.

17. The method of claim 16 wherein said polysaccharide is Chitosan.

18. The method of claim 16 wherein the clot formation accelerator is a thrombogenic agent.

19. The method of claim 15 further comprising drying said gelatin foam hemostasis material matrix above a freezing point temperature.

20. A method for forming a clot formation accelerator loaded hemostasis material, comprising:
heating gelatin granules in water;
adding a cross-linking agent;
mixing a clot formation accelerator to the cross-linking agent and heated gelatin solution; and
drying said clot formation accelerator mixture at a temperature above a freezing point temperature to form said hemostasis material,
wherein said clot formation accelerator is substantially dispersed throughout said hemostasis material.

21. The method of claim 20 wherein said heating further comprises adding a polysaccharide.

22. The method of claim 21 wherein said polysaccharide is Chitosan.

23. The method of claim 21 wherein the clot formation accelerator is a thrombogenic agent.

24. An apparatus for forming a clot formation accelerator loaded hemostasis material, comprising:
means for heating gelatin granules in water;
means for adding a cross-linking agent;
means for mixing a clot formation accelerator to the cross-linking agent and heated gelatin solution; and
means for adding air to form a gelatin foam hemostasis material matrix,
wherein said clot formation accelerator is substantially dispersed throughout said hemostasis material.

25. The apparatus of claim 24 wherein said means for dissolving further comprises adding a polysaccharide.

26. The apparatus of claim 25 wherein said polysaccharide is Chitosan.

27. The apparatus of claim 25 wherein the clot formation accelerator is a thrombogenic agent.

28. The apparatus of claim 24 further comprising means for drying said gelatin foam hemostasis material matrix above a freezing point temperature.

29. An apparatus for forming a clot formation accelerator loaded hemostasis material, comprising:
means for heating gelatin granules in water;
means for adding a cross-linking agent;
means for mixing a clot formation accelerator to the cross-linking agent and heated gelatin solution; and
means for drying said clot formation accelerator mixture at a temperature above a freezing point temperature to form said hemostasis material,
wherein said clot formation accelerator is substantially dispersed throughout said hemostasis material.

30. The apparatus of claim 29 wherein said means for heating further comprises adding a polysaccharide.

31. The apparatus of claim 30 wherein said polysaccharide is Chitosan.

32. The apparatus of claim 30 wherein the clot formation accelerator is a thrombogenic agent.
